(19) United States
(12) Patent Application Publication
(10) Pub. No.: US 2020/0378945 A1
(43) Pub. Date: Dec. 3, 2020

(54) ROAD SURFACE DAMAGE DETECTION DEVICE AND ROAD INFORMATION PROVIDING SYSTEM

(71) Applicant: AISIN SEIKI KABUSHIKI KAISHA, Kariya-shi (JP)

(72) Inventors: Shuji YAMAMOTO, Kariya-shi (JP); Toshiro Umemura, Kariya-shi (JP); Chenwei Yu, Kariya-shi (JP)

(73) Assignee: AISIN SEIKI KABUSHIKI KAISHA, Kariya-shi (JP)

(21) Appl. No.: 16/853,830

(22) Filed: Apr. 21, 2020

(30) Foreign Application Priority Data
May 29, 2019 (JP) 2019-100688

(51) Int. Cl. G01N 33/42; G01N 21/88; G01N 21/95; G01N 19/08

(52) U.S. Cl. CPC G01N 33/42; G01N 19/08; G01N 21/95; G01N 21/8851

(57) ABSTRACT

A road surface damage detection device includes: a front-rear acceleration acquisition unit configured to acquire a front-rear directional acceleration of a vehicle during travelling of the vehicle; a position information acquisition unit configured to acquire position information indicating a current position of the vehicle; a damage state acquisition unit configured to acquire road surface damage information which is related to a road surface on which the vehicle travels and in which road surface damage of the road surface detected based on a change state of the acceleration, and the position information corresponding to a position where the road surface damage is detected are associated with each other; and a transmission unit configured to transmit the acquired road surface damage information to an outside of the vehicle.

### CROSS REFERENCE TO RELATED APPLICATIONS

This application is based on and claims priority under 35 U.S.C. § 119 to Japanese Patent Application 2019-100688, filed on May 29, 2019, the entire contents of which are incorporated herein by reference.

### TECHNICAL FIELD

Embodiments of this disclosure relate to a road surface damage detection device and a road information providing system.

### BACKGROUND DISCUSSION

In the related art, there has proposed a device that, when a road surface condition of a road is managed, for example, when a road surface state such as a crack is managed, performs management by a manager or the like visually checking a captured image captured by an on-board camera when actually traveling on the road, and performs image processing on the captured image to detect damage such as the crack. Further, there has proposed a device that collects and outputs information related to road surface damage, when a vehicle travels and there is damage on a road surface, with an attention to a point that a rotation speed of only a wheel that passes through a damaged portion changes compared with other wheels.

Examples of the related art include JP 2018-21375A (Reference 1) and JP 2016-66144A (Reference 2).

However, when the road surface condition is managed by visually checking the captured image or performing the image processing, in some cases, the damage such as the crack cannot be accurately recognized (detected) due to an image capturing state (environment at the time of image capturing), for example, light of an oncoming vehicle, a street light, and a sunshine state. Further, there is a problem that a dedicated imaging device and image analysis device are required, and a system becomes expensive. Further, the road surface damage may be detected by a difference in rotation speed of each wheel, the rotation speed of each wheel tends to change not only when there is road surface damage but also when the vehicle steps on sand or gravel on the road surface or when the vehicle passes through a partially wet road surface, and erroneous detection may be likely to occur.

Thus, a need exists for a road surface damage detection device which is not susceptible to the drawback mentioned above.

### SUMMARY

A road surface damage detection device according to an aspect of this disclosure includes, for example, a front-rear acceleration acquisition unit configured to acquire a front-rear directional acceleration of a vehicle during travelling of the vehicle; a position information acquisition unit configured to acquire position information indicating a current position of the vehicle; a damage state acquisition unit configured to acquire road surface damage information, which is related to a road surface on which the vehicle travels and in which road surface damage of the road surface detected based on a change state of the acceleration, and the position information corresponding to a position where the road surface damage is detected are associated with each other; and a transmission unit configured to transmit the acquired road surface damage information to an outside of the vehicle.

### BRIEF DESCRIPTION OF THE DRAWINGS

The foregoing and additional features and characteristics of this disclosure will become more apparent from the following detailed description considered with the reference to the accompanying drawings, wherein:

FIG. 1 is an exemplary schematic configuration diagram showing a configuration of a road surface damage detection device and a road information providing system connected to the road surface damage detection device via a network according to an embodiment;

FIG. 2 is an exemplary schematic diagram showing a state of a front-rear directional acceleration received by a vehicle (a wheel) when a road surface damage (for example, a crack) occurs, in the road surface damage detection device according to the embodiment;

FIG. 3 is an exemplary schematic diagram showing another state of the front-rear directional acceleration received by the vehicle (the wheel) when the road surface damage (for example, the crack) occurs, in the road surface damage detection device according to the embodiment;

FIG. 4 is an exemplary schematic diagram showing a change amount of the front-rear directional acceleration acquired during travelling and a threshold value for determining whether to consider the road surface damage, in the road surface damage detection device according to the embodiment;

FIG. 5 is an exemplary schematic diagram showing the number of acquisition times of sensor information that can be acquired during travelling of a predetermined distance, in the road surface damage detection device according to the embodiment;

FIG. 6 is an exemplary schematic diagram showing processing of constructing map data based on road surface damage information, and the processing is executed by the road information providing system according to the embodiment;

FIG. 7 is an exemplary flowchart showing generation and transmission of the road surface damage information in the road surface damage detection device according to the embodiment;

FIG. 8 is an exemplary flowchart showing construction of a road surface state database, and the construction is executed by the road information providing system according to the embodiment;

FIG. 9 is an exemplary flowchart showing processing of constructing the road surface state database at coordinates (X, Y) on a map, and the processing is executed by the road information providing system according to the embodiment;

FIG. 10 is an exemplary flowchart showing processing of providing the road surface damage information, and the processing is executed by the road information providing system according to the embodiment;

FIG. 11 is an exemplary schematic configuration diagram showing another configuration of the road surface damage detection device and the road information providing system connected to the road surface damage detection device via the network according to the embodiment;

FIG. 12 is an exemplary flowchart showing generation and transmission of road surface damage related information, and the generation and transmission is executed by the road surface damage detection device according to FIG. 11; and

FIG. 13 is an exemplary flowchart showing generation of road surface damage information and construction of road surface state database, and the generation and construction is executed by the road information providing system according to FIG. 11.

### DETAILED DESCRIPTION

An embodiment disclosed here will be disclosed below. A configuration of the embodiment to be described below, and operations, results, and effects provided by the configuration are examples. This disclosure can be implemented by configurations other than those disclosed in the following embodiment, and can obtain at least one of various effects based on the basic configuration and derivative effects.

A road surface damage detection device according to the present embodiment detects a road surface damage state during travelling, for example, a state such as a “crack”, using an existing sensor for detecting a state (motion) of a vehicle, for example, a sensor (for example, a front-rear acceleration sensor) that detects an acceleration acting on the vehicle. The road surface damage detection device according to the present embodiment transmits road surface damage information based on a detection result to outside of the vehicle (for example, a road information providing system) and allows a user outside the vehicle to use the information. The road information providing system manages the road surface damage information transmitted from the road surface damage detection device. The road information providing system efficiently provides the road surface damage information to the user, for example, provides the road surface damage information in a travelling direction (front side) to a vehicle travelling on a road corresponding to the road surface damage information or a driver of the vehicle, and the road surface damage information is used for driving control and alerting the driver of the vehicle during travelling. The road information providing system provides the road surface damage information to a road manager and the road surface damage information is used as data for road management, road surface repair, mend, and the like.

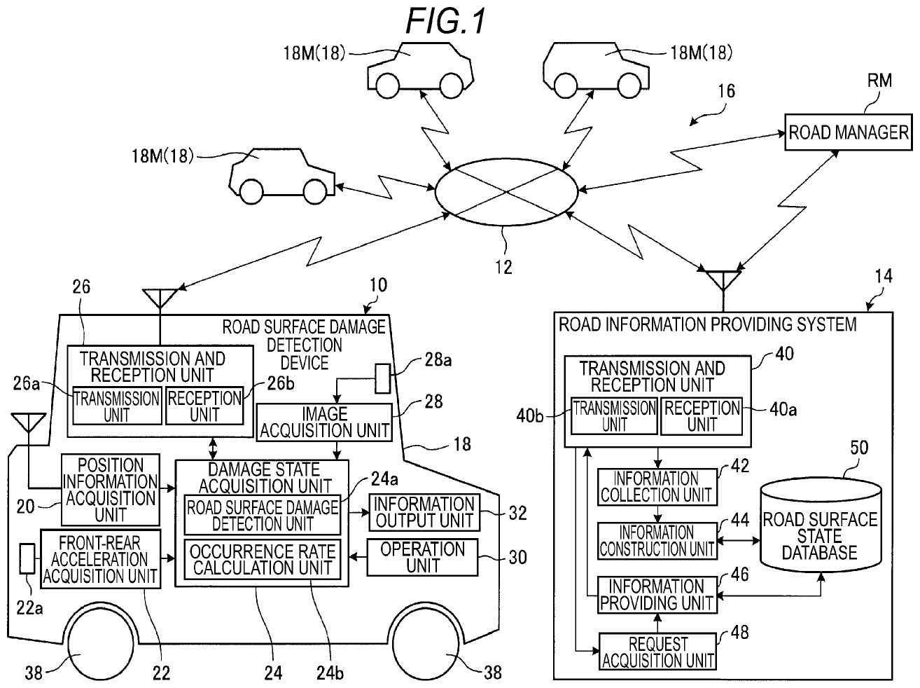

FIG. 1 is an exemplary schematic configuration diagram showing a configuration of an information sharing system 16 including a road surface damage detection device 10 and a road information providing system 14 connected to the road surface damage detection device 10 via a network 12 according to the embodiment. The road surface damage detection device 10 is mounted on a vehicle 18, and sequentially collects data on a road surface as the vehicle 18 travels and transmits the data to the road information providing system 14.

FIG. 1 shows an example of the information sharing system 16 in which road surface damage information (information indicating detection of road surface damage or an existing position) is generated on a road surface damage detection device 10 side mounted on the vehicle 18 and is transmitted to the road information providing system 14.

The road surface damage detection device 10 is implemented by hardware such as a processor and a memory, and each functional module is implemented by the processor reading and executing a program stored in the memory such as a storage unit. The road surface damage detection device 10 includes, for example, a position information acquisition unit 20, a front-rear acceleration acquisition unit 22, a damage state acquisition unit 24, a transmission and reception unit 26, an image acquisition unit 28, an operation unit 30, and an information output unit 32 as functional modules. The position information acquisition unit 20, the front-rear acceleration acquisition unit 22, the damage state acquisition unit 24, the transmission and reception unit 26, the image acquisition unit 28, the operation unit 30, and the information output unit 32 may be implemented by independent hardware. Each functional module is an example, and functions may be integrated or subdivided as long as the same function can be implemented.

The position information acquisition unit 20 acquires position information indicating a current position of the vehicle 18. The position information acquisition unit 20 can acquire the position information of the vehicle 18 from a global positioning system (GPS), for example. The position information acquisition unit 20 may acquire the position information of the vehicle 18 acquired by another system such as a navigation system mounted on the vehicle 18. The position information acquisition unit 20 sequentially provides the acquired position information to the damage state acquisition unit 24.

The front-rear acceleration acquisition unit 22 acquires a front-rear directional acceleration received by the vehicle 18 during travelling. The front-rear acceleration acquisition unit 22 can acquire the front-rear directional acceleration from an acceleration sensor 22a existing in the vehicle 18, for example. The existing acceleration sensor 22a can be, for example, an acceleration sensor used for posture detection or sideslip detection of the vehicle 18, or an acceleration sensor for impact detection used in an airbag system, and acquires the front-rear directional acceleration of the vehicle 18 among the accelerations. The front-rear acceleration acquisition unit 22 may acquire the front-rear directional acceleration from a sensor dedicated to detection of the road surface damage. The front-rear acceleration acquisition unit 22 sequentially provides acquired front-rear directional accelerations to the damage state acquisition unit 24.

The damage state acquisition unit 24 acquires (generates) the road surface damage information which is related to a road surface on which the vehicle 18 travels and in which the road surface damage of the travelled road surface detected based on a change state of the front-rear directional acceleration and the position information corresponding to a position where the road surface damage is detected is associated with each other. In the present embodiment, an example in which the “crack” on the road surface is detected as the road surface damage will be described. The crack on the road surface may cause a reduction in riding comfort of the vehicle 18, damage to a wheel (a tire), an irregular motion of the wheel, and the like. Therefore, when there is a crack or the like that affects the travel, it is desirable to alert the driver or to reduce an influence with a reflection on travelling control of the vehicle 18. It is desirable that the crack (the road surface damage) with a predetermined size or more is promptly repaired (mended) by a road manager RM. For example, it is desirable that the road manager RM acquires the road surface damage information from the information sharing system 16 (for example, via the network 12 or the road information providing system 14) to check a degree of the road surface damage. Therefore, the damage state acquisition unit 24 needs to acquire highly accurate information related to a damaged road surface.

Thus, the damage state acquisition unit 24 of the present embodiment includes a road surface damage detection unit 24a and an occurrence rate calculation unit 24b.

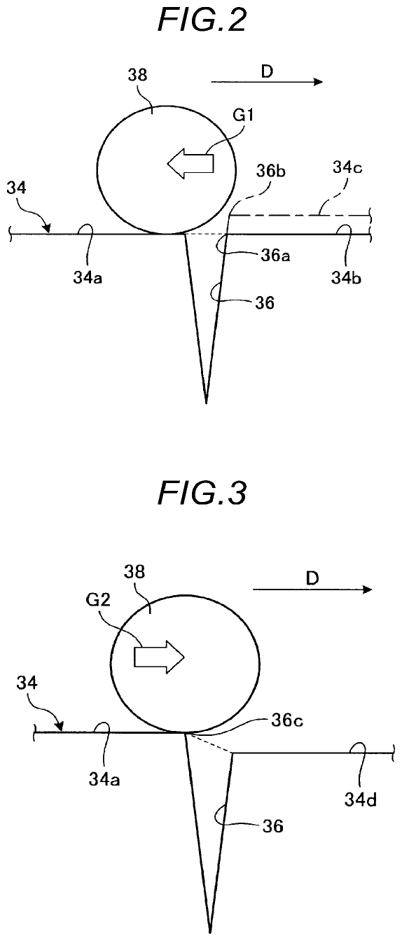

The road surface damage detection unit 24a detects presence or absence of the road surface damage by analyzing the front-rear directional acceleration of the vehicle 18 sequentially acquired by the front-rear acceleration acquisition unit 22. For example, as shown in FIGS. 2 and 3, a case where a road surface damage 36 (for example, a crack) exists on a road surface 34, and a wheel 38 (a tire) of the vehicle 18 passes through the road surface 34 is considered. When the road surface damage 36 occurs on the road surface 34, a magnitude and form of the road surface damage 36 may differ depending on a cause, an environment of a place, or a degree of aging of the road surface damage 36. For example, as shown in FIG. 2, as a form of the road surface damage 36, a front side road surface 34a of the road surface damage 36 and a to-be-passed rear side road surface 34b of the road surface damage 36 may have substantially the same height, or a to-be-passed rear side road surface 34c may be higher than the front side road surface 34a with respect to a travelling direction D of the vehicle 18. Conversely, as shown in FIG. 3, a to-be-passed rear side road surface 34d may be lower than the front side road surface 34a of the road surface damage 36 with respect to the travelling direction D of the vehicle 18. In either case, when the wheel 38 passes through a damaged portion of the road surface 34 during travelling, a front-rear directional acceleration occurs in the vehicle 18 in accordance with a degree of the damage. Thus, the detection of the road surface damage based on the change in the front-rear directional acceleration can implement detection with higher accuracy than the detection of the road surface damage based on a difference in a wheel speed in which a detected value also changes in a road surface state other than the road surface damage.

For example, as shown in FIG. 2, in a case where the wheel 38 passes through the road surface damage 36 in which the front side road surface 34a and the to-be-passed rear side road surface 34b have substantially the same height, when the wheel 38 hits an edge 36a of the to-be-passed rear side road surface 34b, the vehicle 18 decelerates for a moment, and receives an acceleration in a direction of an arrow G1. As a result, the front-rear acceleration acquisition unit 22 in the vehicle 18 receives a forward acceleration in a direction (a rightward direction in the figure) opposite to the direction of the arrow G1 due to inertia. In this case, when a speed of the vehicle 18 is the same, the wider a distance (a width of the crack) between the front side road surface 34a and the to-be-passed rear side road surface 34b of the road surface damage 36, the greater a tendency to receive a large forward acceleration. Similarly, in a case where the wheel 38 passes through the road surface damage 36 in which the to-be-passed rear side road surface 34c is higher than the front side road surface 34a, when the wheel 38 hits an edge 36b of the to-be-passed rear side road surface 34c, the vehicle 18 decelerates for a moment, and receives an acceleration in the direction of the arrow G1. As a result, the front-rear acceleration acquisition unit 22 in the vehicle 18 receives a forward acceleration in the direction (the rightward direction in the figure) opposite to the direction of the arrow G1 due to inertia. In this case, when the speed of the vehicle 18 is the same, the wider a distance between the front side road surface 34a and the to-be-passed rear side road surface 34c of the road surface damage 36 and the greater a difference in height between the front side road surface 34a and the to-be-passed rear side road surface 34c, the greater a tendency to receive a large forward acceleration.

As shown in FIG. 3, in a case where the wheel 38 passes through the road surface damage 36 in which the to-be-passed rear side road surface 34d is lower than the front side road surface 34a, when the wheel 38 hits an edge 36c of the front side road surface 34a, the wheel 38 is separated from the road surface 34, a road surface resistance reduces for a moment, and the vehicle 18 accelerates as the resistance reduces, and receives an acceleration in a direction of an arrow G2. As a result, the front-rear acceleration acquisition unit 22 in the vehicle 18 receives a rearward acceleration in a direction (a leftward direction in the figure) opposite to the direction of the arrow G2 due to inertia. In this case, when the speed of the vehicle 18 is the same, the larger a dynamic friction between the road surface 34 (the front side road surface 34a) with the road surface damage 36 and the wheel 38, the greater a tendency to receive a large rearward acceleration.

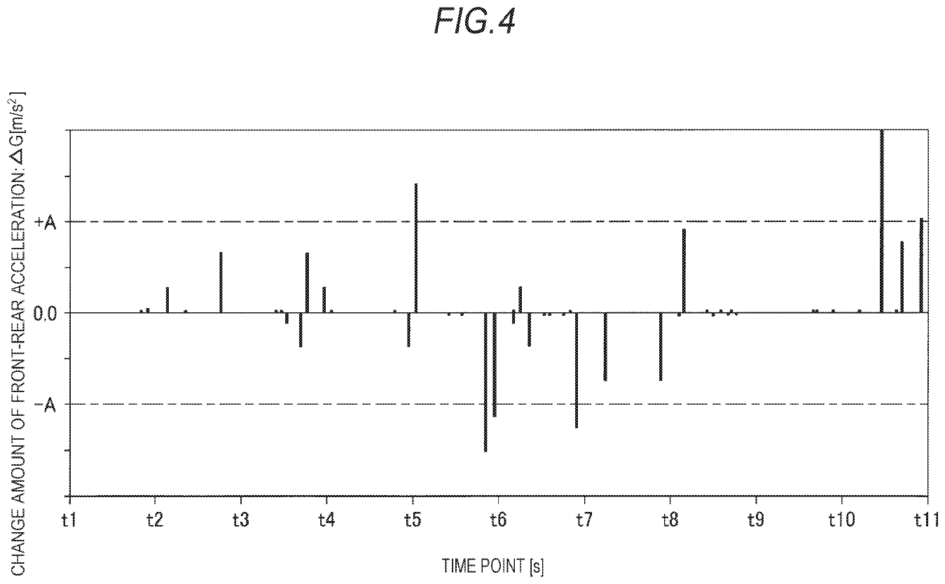

FIG. 4 is an exemplary schematic diagram showing a change amount of the front-rear directional acceleration acquired by the road surface damage detection unit 24a during travelling of the vehicle 18. FIG. 4 shows, as an example, the change amounts of the front-rear directional accelerations from a time point t1 to a time point t11. The road surface damage detection unit 24a acquires the front-rear directional acceleration (sensor information) from the front-rear acceleration acquisition unit 22 in a sampling cycle T. In this case, when the front-rear directional acceleration changes, it can be considered that there is a possibility that the road surface damage (for example, the crack) may exist as described above. A change amount ΔG of the front-rear directional acceleration may be, for example, a value obtained by taking a time difference with respect to the front-rear directional acceleration (sensor information) acquired from the front-rear acceleration acquisition unit 22, or a value obtained by removing a moving average of the acceleration from the acquired front-rear directional acceleration. As described above, the change amount ΔG of the front-rear directional acceleration differs depending on the degree of the road surface damage (for example, the crack). In the present embodiment, when the change amount ΔG of the front-rear directional acceleration is detected, a threshold value is provided in order to identify a road surface damage that requires alerting and repair and a non-road surface damage that does not requires alerting and repair at a current stage. As described above, the front-rear directional acceleration may appear as the forward acceleration or the rearward acceleration depending on the form of the road surface damage. Therefore, as shown in FIG. 4, the road surface damage detection unit 24a sets a threshold value+A and a threshold value −A for the change amount ΔG of the front-rear directional acceleration, and determines the road surface damage (for example, the crack) when the change amount ΔG is equal to or more than the threshold value+A or equal to or less than the threshold value −A. Therefore, when the change amount ΔG is between the threshold value+A and the threshold value −A, the road surface damage (for example, the crack) is determined to be negligible at the current stage. The threshold values+A and −A can be determined in advance by determining the form and magnitude of the road surface damage that requires alerting and repair by a test or the like. The threshold values+A and −A may be appropriately changed in accordance with a required degree of the road surface damage.

Although the vehicle 18 accelerates and decelerates during traveling, an acceleration change and a deceleration change during normal travelling are generally performed slowly, so that an instantaneous change in the acceleration caused by the road surface damage can be identified. Vehicle control information such as a change amount of depression of an accelerator pedal or a brake pedal and control information for automatic acceleration or automatic deceleration (braking) can be acquired from a vehicle 18 side to more clearly identify the change in the acceleration caused by the acceleration and the deceleration during travelling of the vehicle 18 and the change in the acceleration caused by the road surface damage.

When the change amount ΔG that is equal to or more than the threshold value +A or equal to or less than the threshold value −A is detected (the road surface damage), the road surface damage detection unit 24a generates the road surface damage information in which a magnitude of the front-rear directional acceleration serving as information indicating the degree of the road surface damage and the position information (acquired by the position information acquisition unit 20) corresponding to a position indicating the road surface damage is associated with each other. A correspondence between the information indicating the degree of the road surface damage and the position information may be obtained by, for example, attaching time stamps when the position information acquisition unit 20 and the front-rear acceleration acquisition unit 22 acquire these pieces of information and matching these pieces of information.

The occurrence rate calculation unit 24b calculates an occurrence rate of the road surface damage for a predetermined travelled range (a predetermined travel distance L). A vehicle speed of the vehicle 18 at a certain time point (for example, the current time point t) and at a position (position coordinates (x₍ₜ₎, y₍ₜ₎) in the position information) is set to V₍ₜ₎. In this case, the occurrence rate of the road surface damage can be calculated based on the number of times of detection of the road surface damage when the vehicle moves to the position coordinates (x₍ₜ₎, y₍ₜ₎) for the predetermined travel distance L (for example, a fixed value).

Here, as shown in FIG. 5, the occurrence rate calculation unit 24b can acquire the sensor information (acceleration information acquired by the front-rear acceleration acquisition unit 22) indicating the front-rear directional acceleration at the sampling cycle T while the vehicle 18 advances for the predetermined travel distance L. In this case, it can be considered that the vehicle 18 at the current time point t with the position coordinates (x₍ₜ₎, y₍ₜ₎), and the vehicle speed V₍ₜ₎ advances for a distance ΔL (=T·V_{(t-T)}) from position coordinates (x_{(t-T)}, y_{(t-T)}) at which the sensor information of one previous time (t−T) and a vehicle speed V_{(t-T)} is obtained. Further, when the road surface damage is detected at a certain sampling cycle T, it can be considered that the road surface damage exists while the vehicle advances for the distance ΔL (=T−V_{(t-T)}). That is, the distance ΔL (=T−V_{(t-T)}) can be considered as a damage section E in which the road surface damage (for example, the crack) exists. As shown in FIG. 5, in the predetermined travel distance L up to that time point t, the number of times n that the sensor information can be acquired can be obtained, as shown in the following Formula 1, as a value that maximizes a sum of the distances ΔL advanced from a time point n times before with respect to the predetermined travel distance L. For example, assuming that the vehicle speed changes during the predetermined travel distance L, the faster the speed, the smaller the number of times n that the sensor information can be acquired, and the lower the speed, the larger the number of times n that the sensor information can be acquired.

$${\sum }_{i = 1}^{n} T \cdot {V}_{( t - i \cdot T )} \leq L \qquad ( Formula 1 )$$

The occurrence rate calculation unit 24b calculates an occurrence rate of the road surface damage in a travel section that is traced back from the current time point t by the predetermined travel distance L. As described above, based on the current time point t, the distance ΔL from a timing (t−i·T) when the road surface damage is detected to the next timing can be expressed by the distance ΔL=T−V_{t-i·T)} by using the vehicle speed V_{(t-i·T)}. Further, the distance ΔL from the timing when the road surface damage is detected can be considered as the damage section E. Therefore, as shown in the following Formula 2, by dividing the sum of the distances ΔL=T·V_{(t-i·T)} by the predetermined travel distance L each time the road surface damage is detected, an occurrence rate p₍ₜ₎ of the road surface damage at the current time point t in the predetermined travel distance L can be calculated.

$${p}_{( t )} = \frac{{\sum }_{i : ROAD SURFACE DAMAGE DETECTION} T \cdot {V}_{( t - i \cdot T )}}{L} \qquad ( Formula 2 )$$

The damage state acquisition unit 24 generates road surface damage information P_{c(t)} by associating the occurrence rate p₍ₜ₎ of the road surface damage at the current time point t with the position coordinates (x₍ₜ₎, y₍ₜ₎) on a map. For example, information shown by the road surface damage information P_{c(t)}=[p₍ₜ₎, x₍ₜ₎, y₍ₜ₎] is generated. The subscript “c” indicates that information is road surface damage information of a certain vehicle c.

The vehicle speed of the vehicle 18 used in calculating the occurrence rate p₍ₜ₎ of the road surface damage at the current time point t may be a constant value while travelling for the predetermined travel distance L. In this case, the occurrence rate p₍ₜ₎ of the road surface damage can be calculated based on how many times the road surface damage is detected with respect to the number of times n that the sensor information is acquired while travelling for the predetermined travel distance L. In this case, a processing load in the damage state acquisition unit 24 can be reduced.

The transmission and reception unit 26 includes a transmission unit 26a and a reception unit 26b. The transmission unit 26a transmits the road surface damage information acquired (generated) by the damage state acquisition unit 24 to the road information providing system 14 outside the vehicle 18, for example, via the network 12. In another embodiment, the road surface damage information may be transmitted directly to other vehicles. The reception unit 26b appropriately receives the road surface damage information (information of a road surface state database, which will be described later) accumulated in the road information providing system 14. For example, the road surface damage information related to a predetermined road (a road surface in the travelling direction) on which the vehicle 18 (own vehicle) is going to travel can be received, and vehicle control can be performed or the driver can be alerted. In another embodiment, the road surface damage information generated by other vehicles may be received.

The image acquisition unit 28 acquires a captured image from an image capturing unit 28a that captures an image of the road surface during travelling of the vehicle 18. The image capturing unit 28a is, for example, a digital camera incorporating an imaging element such as a charge coupled device (CCD) or a CMOS image sensor (CIS). The image capturing unit 28a can output video data (captured image data) at a predetermined frame rate. The image capturing unit 28a includes, for example, a wide-angle lens or a fisheye lens, and can capture an image, for example, in a range of 140° to 220° in a horizontal direction. An image of a position of the road surface damage can be captured in detail by adjusting timing for capturing an image, even without using the wide-angle lens. An image of a road surface condition and a surrounding condition can be imaged with an optical axis of the image capturing unit 28a obliquely downward. An image acquired by the image acquisition unit 28 and the road surface damage information acquired by the damage state acquisition unit 24 is associated with each other, and is transmitted via the transmission unit 26a. That is, the captured image corresponding to a position where the road surface damage is detected and the road surface damage information is associated with each other, and when the road surface damage information is used, it is possible to easily grasp the degree of the road surface damage, an actual damage state, and the like. By attaching a time stamp to the captured image, it is possible to easily associate the road surface damage information (the position of the road surface damage) with the captured image.

In the case of FIG. 1, the image capturing unit 28a is disposed inside a front window so as to capture an image of the travelling direction of the vehicle 18 (a front side of the vehicle). In another embodiment, an installation position of the image capturing unit 28a can be appropriately changed as long as a road surface through which each wheel 38 of the vehicle 18 passes in an image capturing range can be included and a correspondence between the position of the road surface damage and the position shown by the captured image can be ensured. For example, the image capturing unit 28a may be provided at a position where an image of a rear side of the vehicle 18 is captured. The captured image may be an imaged captured by an image capturing unit disposed on a lateral side of the vehicle 18, for example, a side mirror. The captured image acquired by the image acquisition unit 28 may be acquired from an image capturing unit provided for generating the road surface damage information, or may be acquired from an image capturing unit provided for a monitoring system that monitors surroundings of the vehicle 18. The captured image including the road surface may be acquired from a drive recorder.

For example, when the road surface damage information is shared between the vehicle 18 and other vehicles 18M (in FIG. 1, three are shown for simplicity) or between the vehicle 18 and the road information providing system 14, the operation unit 30 receives a request operation when the driver of the vehicle 18 requests acquisition of the road surface damage information generated outside. A request signal is transmitted to, for example, the road information providing system 14 via the damage state acquisition unit 24, the transmission unit 26a, and the network 12, and as a response, the reception unit 26b acquires the road surface damage information corresponding to the request from the road information providing system 14. The acquired road surface damage information is output from the information output unit 32 via the damage state acquisition unit 24. When the road surface damage information is requested, it is possible to specify a current position of the vehicle 18, a road name that can be identified at the current position, and a travelling direction of the own vehicle. When a travel route is guided using a navigation system or the like, the road surface damage information requested based on the current position of the vehicle 18 and the travel route may be specified. When the road surface damage information is used for travelling control of the vehicle 18, the processor or the like mounted on the vehicle 18 may automatically request the road surface damage information.

The information output unit 32 alerts the driver when the road damage exists by displaying contents of the road surface damage information transmitted from the outside and acquired by the transmission unit 26a in a state where the driver can visually recognize, or presenting the information by voice or the like. For example, on a map displayed on a display device, the position of the road surface damage can be displayed, or the degree of the road surface damage (danger level) can be displayed. The existence of the road surface damage in the front side or the degree of the road surface damage (danger level) may be notified via a speaker or the like. In a case of these information outputs, a specific distance to the position of the road surface damage may be presented.

In FIG. 1, other vehicles 18M include a road surface damage detection device 10 similar to the vehicle 18. The vehicle 18 (other vehicles 18M) transmits the road surface damage information acquired via the network 12 to the road information providing system 14, and forms the information sharing system 16 that acquires necessary road surface damage information from the road information providing system 14. In another embodiment, the vehicle 18 and the other vehicles 18M may communicate with each other via the network 12, and may directly transmit and receive the road surface damage information. In another embodiment, other vehicles 18M that do not include the road surface damage detection device 10 may receive the road surface damage information via the network 12 and transmit the information to another vehicle 18M that do not include the road surface damage detection device 10. That is, the road surface damage information can also be used by other vehicles 18M that do not include the road surface damage detection device 10, and the road surface damage information may be shared between other vehicles 18M that do not include the road surface damage detection device 10.

Next, a configuration of the road information providing system 14 will be described. The road information providing system 14 can be implemented by, for example, a personal computer having hardware such as a processor and a memory. More specifically, each functional module is implemented by the processor of the road information providing system 14 reading and executing a program stored in the memory such as a storage unit. The various functional modules include, for example, a transmission and reception unit 40, an information collection unit 42, an information construction unit 44, an information providing unit 46, and a request acquisition unit 48. The transmission and reception unit 40, the information collection unit 42, the information construction unit 44, the information providing unit 46, and the request acquisition unit 48 may be implemented by independent hardware. Each functional module is an example, and functions may be integrated or subdivided as long as the same function can be implemented, and may be implemented by, for example, a cloud.

The transmission and reception unit 40 includes a reception unit 40a and a transmission unit 40b. The reception unit 40a sequentially receives the road surface damage information acquired related to the road on which the vehicle 18 travels. The reception unit 40a receives a request signal for the road surface damage information requested by the vehicle 18 and a request signal for the road surface damage information requested by the road manager RM. The transmission unit 40b transmits the road surface damage information corresponding to the request signal acquired from the vehicle 18, the road manager RM, and the like to the vehicle 18, the road manager RM, and the like that transmit the request signal.

The information collection unit 42 collects the road surface damage information transmitted from at least one of the road surface damage detection devices 10 and received by the reception unit 40a. The information collection unit 42 may specify a specific road or region to collectively collect the road surface damage information from a specific vehicle 18 travelling on the road or to collect the road surface damage information from a plurality of vehicles 18 travelling on the road. By collecting the road surface damage information corresponding to the same region, for example, the position coordinates (x₍ₜ₎, y₍ₜ₎) indicating a specific position, from the plurality of vehicles 18, accuracy of the road surface damage information corresponding to the position coordinates (x₍ₜ₎, y₍ₜ₎) can be improved.

The information construction unit 44 aggregates the road surface damage information collected by the information collection unit 42 for each road to construct a road surface state database 50. As described above, the road surface damage information includes the position coordinates at which the road surface damage is detected, and can specify a detailed position on map data. On the other hand, the driver or the road manager RM who actually uses the road surface damage information may be able to easily deal with the road surface damage by the provided presence or absence of the road surface damage and the degree of the damage based on macro-displayed information (information shown in a wide range with a small scale). For example, when the road surface damage information is used for alerting the driver, rather than alerting with the individual road surface damage by notifying the position of the individual road surface damage, it is easier to use the road surface damage information so as to give a notification before a region where a lot of road surface damages exist is reached and alert the driver for a driving operation while passing through the region. As compared with the case of notifying the individual road surface damage, it is possible to reduce an excessive notification and troublesome feeling of the driver. Similarly, when the road manager RM uses the road surface damage information to determine whether the road is to be repaired or mended, work efficiency may be improved by excluding serious road surface damage that requires immediate repair (mend), and determining a repair range and then a priority of repair.

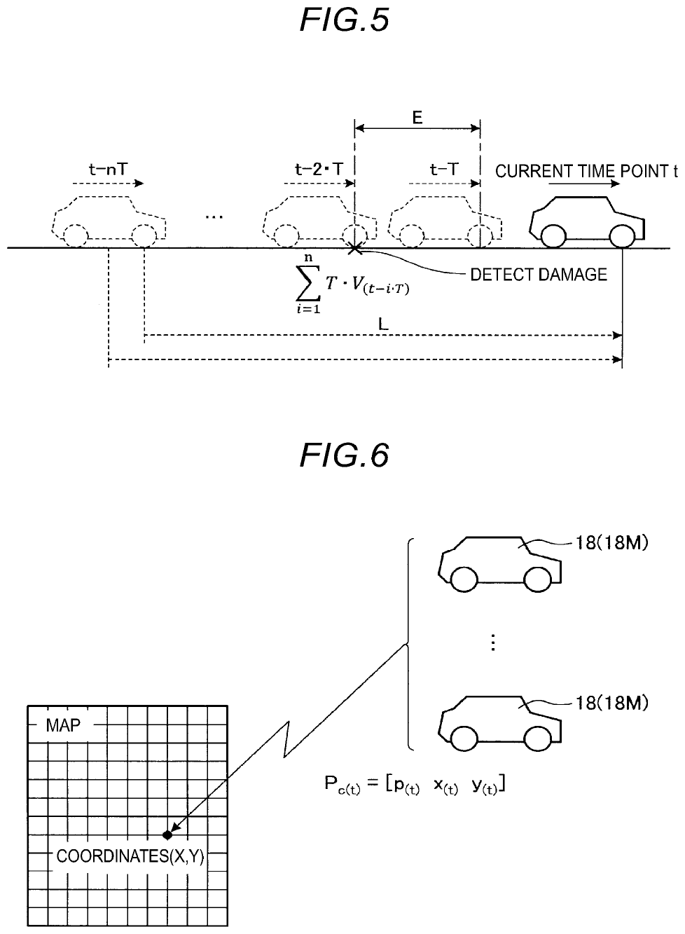

FIG. 6 is an exemplary schematic diagram showing construction processing of the road surface damage information on the map data based on the road surface damage information, and the construction processing is executed by the road information providing system 14. As shown in FIG. 6, a case is considered where the position coordinates (x₍ₜ₎, y₍ₜ₎) in a plurality of pieces of the road surface damage information P_{c(t)}=[P₍ₜ₎, x₍ₜ₎, y₍ₜ₎] collected by the information collection unit 42 belong to coordinates (X, Y) on a map with a practical scale. For example, the plurality of pieces of the road surface damage information P_{c(t)} are included in a 10 m×10 m region (grid) defined by the coordinates (X, Y). In this case, the road surface damage information P_{c(t)} may be information transmitted from the same vehicle 18, or may be information transmitted from the vehicle 18 or other vehicles 18M. The information construction unit 44 acquires an average value of occurrence rates p₍ₜ₎ of the road surface damage included in the road surface damage information P_{c(t)} acquired in all the vehicles 18 (other vehicles 18M) included in the region (grid) defined by the coordinates (X, Y) as shown in the following Formula 3, thereby acquiring an occurrence rate P(X, Y) of the road surface damage at the coordinates (X, Y). In Formula 3, N is the number of samples of the road surface damage information of all the vehicles 18 belonging to the coordinates (X, Y). α is an experimental value, which is a constant obtained for correcting a reduction in a calculation value of an intra-grid probability by sampling, and can be determined in advance based on a test or the like.

$$P ( X , Y ) = α \cdot \frac{{\sum }_{C , ( {x}_{( t )} , {y}_{( t )} ) ∈ ( X , Y )} {p}_{t}}{N} \qquad ( Formula 3 )$$

The information construction unit 44 acquires the occurrence rate P(X, Y) of the road surface damage for each region (grid) defined by the coordinates (X, Y), and constructs the road surface state database 50. As described above, the road surface state database 50 may be constructed for each region (grid) defined by the coordinates (X, Y), or, for example, the road surface state database 50 indicating the road surface damage for each road may be constructed. By constructing the road surface state database 50 using the road surface damage information P_{c(t)}=[p₍ₜ₎, x₍ₜ₎, y_{c(t)}] acquired by the plurality of vehicles 18 for the region defined by the coordinates (X, Y), it is possible to improve accuracy of information indicating the road surface damage in the region defined by the coordinates (X, Y). When the road surface state database 50 is constructed by the road surface damage information from a single vehicle 18, depending on an acquisition cycle of the road surface damage information of the road surface damage detection unit 24a and a transmission cycle of the transmission unit 26a, detection omission of the road surface damage may occur. On the other hand, when the road surface damage is detected by the vehicle 18 travelling within the same region at different timings, the acquisition cycle and transmission cycle of the road surface damage information vary. As a result, the detection omission of the road surface damage can be reduced. In this case, the road surface damage information obtained as a result of the same vehicle 18 travelling in the same place at different timings may be used, or the road surface damage information obtained as a result of other vehicles 18 travelling in the same place may be used.

The information providing unit 46 acquires requested road surface damage information from the road surface state database 50 in accordance with a request signal from a user side (such as the driver or the road manager RM) received by the request acquisition unit 48 via the reception unit 40a. Further, the information providing unit 46 returns the requested road surface damage information to a request source that transmitted the request signal via the transmission unit 40b. For example, when the road surface damage information related to a region in the front side of the travelling vehicle 18 is requested, the information providing unit 46 selects a corresponding region (for example, a range with a radius of 200 m in the front side with the current position of the vehicle 18 as a center) from the road surface state database 50 and transmits information in the selected region. When the road surface damage information related to the road on which the vehicle 18 travels is requested, the information providing unit 46 selects the road surface damage information related to the front side of the vehicle 18, for example, 200 m front of the vehicle 18 on a corresponding road from the road surface state database 50 and transmits the selected road surface damage information.

When the request acquisition unit 48 receives a request for the detailed road surface damage information, the information providing unit 46 may select micro road surface damage information in addition to or instead of macro road surface damage information of the corresponding region from the road surface state database 50 and transmit the selected macro road surface damage information to the request source. For example, in order for the vehicle 18 to perform automatic travelling control or for the road manager RM to specify a repair position on the road surface, the detailed road surface damage information may be requested. Thus, the information providing unit 46 can use the road surface damage information more practically and efficiently by appropriately changing contents to be selected from the road surface state database 50 in accordance with an application of the road surface damage information.

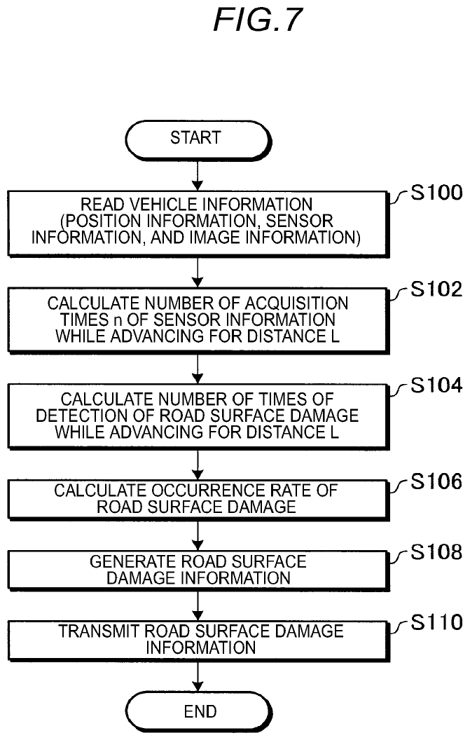

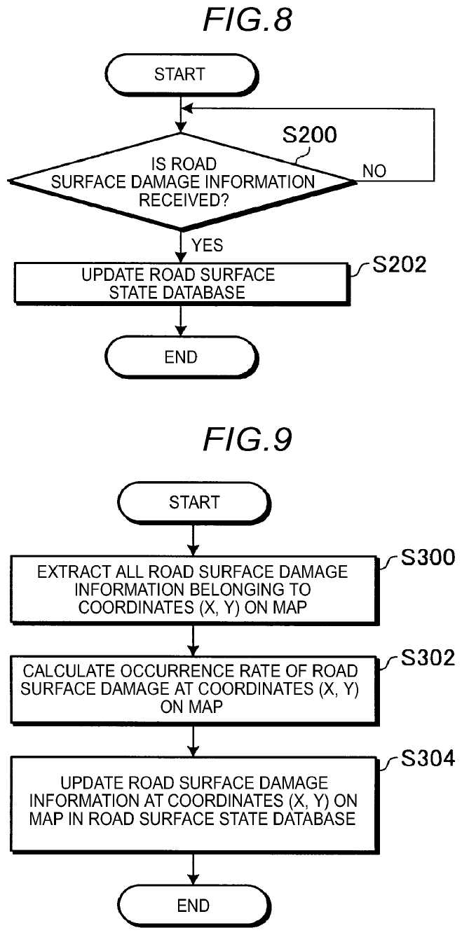

An example of a flow of processing in the road surface damage detection device 10 and the road information providing system 14 (the information sharing system 16) configured as described above will be described with reference to flowcharts of FIGS. 7 to 10. FIG. 7 is a flowchart showing an example of a flow of processing of the road surface damage detection device 10 executed on the vehicle 18 side. FIGS. 8 to 10 are flowcharts showing an example of flows of processing executed on a road information providing system 14 side.

First, as shown in the flowchart of FIG. 7, the damage state acquisition unit 24 reads vehicle information at a predetermined processing cycle during travelling (S100). The vehicle information includes, for example, the position information indicating the current position of the vehicle 18 and acquired by the position information acquisition unit 20, the sensor information acquired by the front-rear acceleration acquisition unit 22, that is, the front-rear directional acceleration indicating the road surface damage, image information (the captured image) acquired by the image acquisition unit 28 and corresponding to the position indicated by the position information and to the position of the road surface damage indicated by the sensor information. As described above, the image information corresponding to the position of the road surface damage is mainly for facilitating visual understanding of the degree and position of the road surface damage, and reading of the image information may be omitted. As described with reference to FIG. 5, the occurrence rate calculation unit 24b calculates the number of acquisition times n of the sensor information while the vehicle advances for the predetermined travel distance L (S102). The road surface damage detection unit 24a, while the vehicle advances for the predetermined travel distance L, calculates the number of times of detection of the road surface damage (S104), and calculates the occurrence rate p₍ₜ₎ of the road surface damage (S106).

Further, the damage state acquisition unit 24 generates the road surface damage information P_{c(t)}=[p₍ₜ₎, x₍ₜ₎, y₍ₜ₎] by associating the occurrence rate p₍ₜ₎ of the road surface damage at the current time point t with the position coordinates (x₍ₜ₎, y₍ₜ₎) on the map (S108). When the road surface damage information P_{c(t)} is generated, the damage state acquisition unit 24 associates the captured image corresponding to the position of the road surface damage acquired by the image acquisition unit 28 with the road surface damage information P_{c(t)}.

The transmission unit 26a sequentially transmits the road surface damage information P_{c(t)} generated by the damage state acquisition unit 24 to the road information providing system 14 via the network 12 (S110), and ends this flow.

Next, processing of the road information providing system 14 will be described with reference to the flowcharts of FIGS. 8 to 10. As shown in the flowchart of FIG. 8, the information collection unit 42 of the road information providing system 14 monitors whether the reception unit 40a receives the road surface damage information P_{c(t)} from the vehicle 18 (No in S200). Further, when the road surface damage information P_{c(t)} is received (Yes in S200), the information collection unit 42 sequentially collects the road surface damage information P_{c(t)}, and the information construction unit 44 updates the road surface state database 50 (S202).

When a plurality of pieces of road surface damage information P_{c(t)} in a predetermined region on the map is provided from a single vehicle 18 or a plurality of pieces of road surface damage information P_{c(t)} in a predetermined region on the map is provided from a plurality of vehicles 18, the information construction unit 44 further executes data construction for improving the accuracy of the road surface damage information in the region.

For example, as shown in FIG. 9, the information construction unit 44 extracts all road surface damage information P_{c(t)} belonging to the coordinates (X, Y) on the map from the plurality of pieces of road surface damage information P_{c(t)} collected by the information collection unit 42 (S300). The information construction unit 44 calculates the occurrence rate P(X, Y) of the road surface damage at the coordinates (X, Y) on the map in accordance with Formula 3 described above (S302). Further, the information construction unit 44 constructs the road surface damage information at the coordinates (X, Y) in the road surface state database 50 based on the plurality of pieces of road surface damage information P_{c(t)} (S304). As a result, the accuracy of the road surface damage information at the coordinates (X, Y) can be improved. When constructing the road surface state database 50, the information construction unit 44 may rank the degree of the road surface damage and attach rank information. For example, when the road surface damage information is provided to the driver of the vehicle 18 or the road manager RM by displaying information on the map, for example, color coding may be performed for each rank so as to be displayed. An alert mark or the like in accordance with the rank may be displayed. That is, the “danger level” corresponding to the road surface damage may be displayed.

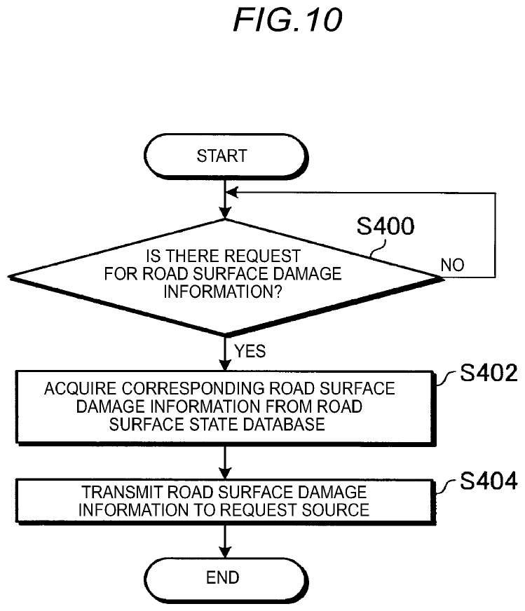

Next, an example of the processing of the road information providing system 14 when a request for the road surface damage information is received from the driver of the vehicle 18 or the road manager RM will be described with reference to FIG. 10.

The request acquisition unit 48 constantly checks whether there is a request for the road surface damage information via the reception unit 40a (No in S400). When the request acquisition unit 48 receives a request for the road surface damage information (Yes in S400), the information providing unit 46 acquires the road surface damage information of a position (a region or a road) corresponding to received request contents, from the road surface state database 50 (S402). Further, the information providing unit 46 transmits the acquired road surface damage information to the vehicle 18 or the road manager RM that is a request source of the road surface damage information via the transmission unit 40b (S404).

As described above, in the information sharing system 16 shown in FIG. 1, the road surface damage detection device 10 mounted on the vehicle 18 detects the front-rear directional acceleration acting on the vehicle 18 by using the existing sensor in the vehicle 18, and generates the road surface damage information. Then, the road surface damage detection device 10 transmits the generated road surface damage information to the road information providing system 14 via the network 12. Further, the road information providing system 14 constructs the road surface state database 50 based on the road surface damage information transmitted from the vehicle 18. When the road information providing system 14 is requested to provide the road surface damage information by the vehicle 18 or the driver, the road manager RM, and the like, the road information providing system 14 selects the road surface damage information corresponding to the request from the road surface state database 50 and provides the selected information. As a result, the information sharing system 16 can provide the latest road surface damage information to the vehicle 18 or the driver, and it is possible to execute an optimum travelling control of the vehicle 18 and an optimum alert to the driver. It is possible to provide the latest road surface damage information to the road manager RM, and to easily perform efficient road management.

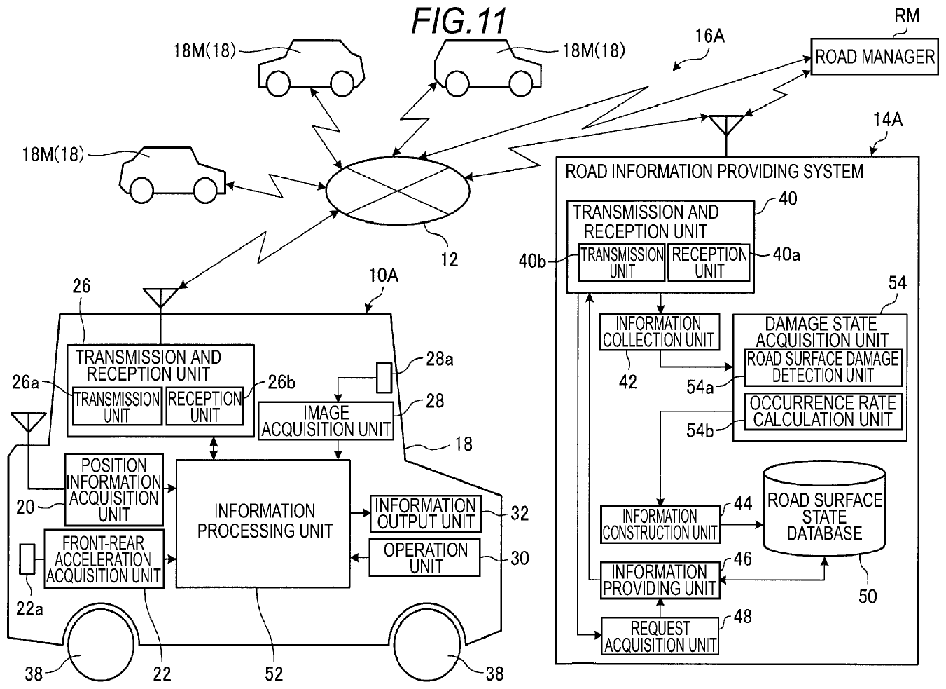

FIG. 11 is an exemplary schematic configuration diagram showing another configuration of a road surface damage detection device 10A and a road information providing system 14A (an information sharing system 16A). The road surface damage detection device 10 shown in FIG. 1 is configured to generate the road surface damage information on the vehicle 18 side and transmit the information to the road information providing system 14, whereas information necessary for generating the road surface damage information is collected on a road surface damage detection device 10A side and transmitted to a road information providing system 14A side as shown in FIG. 11. The road information providing system 14A generates the road surface damage information based on the received various kinds of information to construct the road surface state database 50. That is, the information sharing system 16A of FIG. 11 and the information sharing system 16 of FIG. 1 have substantially the same configuration except for a place where the road surface damage information is generated. Therefore, in the information sharing system 16A of FIG. 11 and the information sharing system 16 of FIG. 1, the same components are denoted by the same reference numerals, and a detailed description thereof is omitted.

The road surface damage detection device 10A is implemented by hardware such as a processor and a memory, and each functional module is implemented by the processor reading and executing a program stored in the memory such as a storage unit. The road surface damage detection device 10A includes, for example, the position information acquisition unit 20, the front-rear acceleration acquisition unit 22, the transmission and reception unit 26, the image acquisition unit 28, the operation unit 30, the information output unit 32, and an information processing unit 52 as functional modules. The position information acquisition unit 20, the front-rear acceleration acquisition unit 22, the transmission and reception unit 26, the image acquisition unit 28, the operation unit 30, the information output unit 32, and the information processing unit 52 may be implemented by independent hardware. Each functional module is an example, and functions may be integrated or subdivided as long as the same function can be implemented.

The position information acquisition unit 20 acquires position information indicating the current position of the vehicle 18. The position information acquisition unit 20 can acquire the position information of the vehicle 18 from the GPS, for example. The position information acquisition unit 20 may acquire the current position information of the vehicle 18 acquired by another system such as a navigation system mounted on the vehicle 18. The position information acquisition unit 20 sequentially provides the acquired position information to the information processing unit 52.

The front-rear acceleration acquisition unit 22 acquires a front-rear directional acceleration received by the vehicle 18 during travelling. The front-rear acceleration acquisition unit 22 can acquire the front-rear directional acceleration from the acceleration sensor 22a existing in the vehicle 18, for example. The existing acceleration sensor 22a can be, for example, an acceleration sensor used for posture detection or sideslip detection of the vehicle 18, or an acceleration sensor for impact detection used in an airbag system, and acquires the front-rear directional acceleration of the vehicle 18 among the accelerations. The front-rear acceleration acquisition unit 22 may acquire the front-rear directional acceleration from a sensor dedicated to the detection of the road surface damage. The front-rear acceleration acquisition unit 22 sequentially provides the acquired front-rear directional accelerations to the information processing unit 52.

The image acquisition unit 28 acquires a captured image from the image capturing unit 28a that captures an image of the road surface during travelling of the vehicle 18. The image acquisition unit 28 sequentially provides the acquired captured images to the information processing unit 52.

The position information acquisition unit 20, the front-rear acceleration acquisition unit 22, and the image acquisition unit 28 attach time stamps or the like to the acquired information, and individual pieces of information can be temporally associated with one another. The information processing unit 52 associates the position information acquired by the position information acquisition unit 20, the road surface damage information acquired by the damage state acquisition unit 24, and the captured image acquired by the image acquisition unit 28 one another based on the time stamps attached to the respective pieces of information to obtain road surface damage related information. Further, the transmission unit 26a transmits the road surface damage related information temporally associated by the information processing unit 52 to the road information providing system 14A via the network 12. In another embodiment, the position information, the damage information, and the captured image to which the corresponding time stamps or the like are attached may be individually transmitted to the road information providing system 14A, and respective pieces of information may be associated with one another on the information providing system 14A side. In this case, the information processing unit 52 may be omitted, and the position information acquisition unit 20, the front-rear acceleration acquisition unit 22, and the image acquisition unit 28 may transmit information via the transmission unit 26a.

The operation unit 30 receives a request operation when the driver of the vehicle 18 requests acquisition of the road surface damage information from outside. A request signal is transmitted to the road information providing system 14A via the information processing unit 52, the transmission unit 26a, and the network 12, and as a response, the reception unit 26b acquires road surface damage information corresponding to the request from the road information providing system 14A. The acquired road surface damage information is output from the information output unit 32 via the information processing unit 52.

Thus, the road surface damage detection device 10A performs simple processing of acquiring information indicating the presence or absence and the position of the road surface damage as the road surface damage related information, and transmitting information to the road information providing system 14A. As a result, the processing load of the road surface damage detection device 10A can be reduced, and a high-performance processor is not required, which can contribute to cost reduction. That is, a hurdle for mounting the road surface damage detection device 10A to each vehicle 18 can be reduced, which can contribute to the spread.

Next, a configuration of the road information providing system 14A will be described. The road information providing system 14A can be implemented by, for example, a personal computer having hardware such as a processor and a memory. More specifically, each functional module is implemented by the processor of the road information providing system 14A reading and executing a program stored in the memory such as a storage unit. The various functional modules include, for example, the transmission and reception unit 40, the information collection unit 42, the information construction unit 44, the information providing unit 46, the request acquisition unit 48, and a damage state acquisition unit 54. The transmission and reception unit 40, the information collection unit 42, the information construction unit 44, the information providing unit 46, the request acquisition unit 48, and the damage state acquisition unit 54 may be implemented by independent hardware. Each functional module is an example, and functions may be integrated or subdivided as long as the same function can be implemented.

The transmission and reception unit 40 includes the reception unit 40a and the transmission unit 40b. The reception unit 40a acquires the road surface damage related information indicating the presence or absence and the position of the road surface damage, such as the acquired position information, front-rear directional acceleration, and image information related to the road on which the vehicle 18 travels, and provides the road surface damage related information to the information collection unit 42. The transmission unit 40b transmits the road surface damage information corresponding to the request signal acquired from the vehicle 18, the road manager RM, and the like to the vehicle 18, the road manager RM, and the like that transmit the request signal.

The information collection unit 42 collects the road surface damage related information (the position information, the front-rear directional acceleration, and the image information) related to the road surface damage, received by the reception unit 40a and transmitted from at least one of the road surface damage detection devices 10A, and provides the road surface damage related information to the damage state acquisition unit 54. The information collection unit 42 may specify a specific road to collectively collect the road surface damage related information from a specific vehicle 18 travelling on the road or collect the road surface damage related information from a plurality of vehicles 18 travelling on the road. By collecting the road surface damage related information corresponding to the same region, for example, the position coordinates (x₍ₜ₎, y₍ₜ₎) indicating a specific position, from a plurality of vehicles 18, the damage state acquisition unit 54 can improve the accuracy corresponding to the position coordinates (x₍ₜ₎, y₍ₜ₎) of the generated road surface damage information.

The damage state acquisition unit 54 includes a road surface damage detection unit 54a and an occurrence rate calculation unit 54b, and performs similar processing as the detection processing of the road surface damage and the calculation processing of the occurrence rate executed on the vehicle 18 side in the road surface damage detection device 10 of FIG. 1. Processing executed by the road surface damage detection unit 54a and the occurrence rate calculation unit 54b is substantially the same as the road surface damage detection unit 24a and the occurrence rate calculation unit 24b in the road surface damage detection device 10, and a detailed description thereof is omitted.

The road surface damage information and the occurrence rate generated by the damage state acquisition unit 54 is provided to the information construction unit 44, and the information construction unit 44 constructs the road surface state database 50. The information providing unit 46 selects, from the road surface state database 50, the road surface damage information corresponding to a request signal from the vehicle 18 side or a request signal from the road manager RM acquired by the request acquisition unit 48, and transmits the road surface damage information to the request source via the transmission unit 40b.

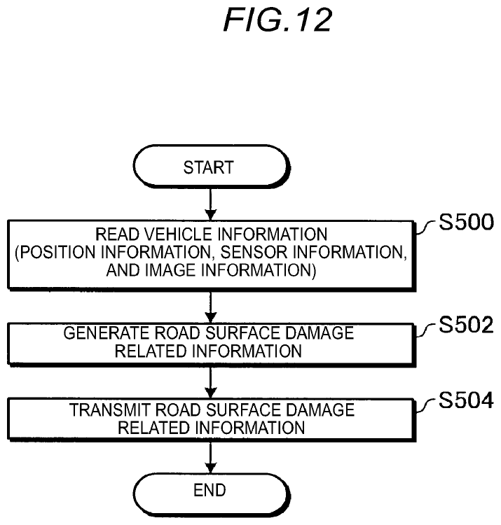

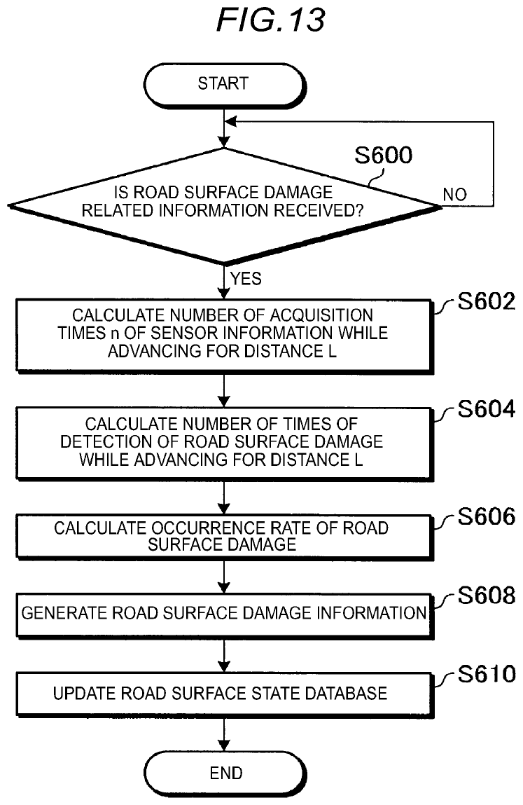

An example of flows of processing in the road surface damage detection device 10A and the road information providing system 14A (the information sharing system 16A) configured as described above will be described with reference to flowcharts of FIGS. 12 and 13. FIG. 12 is a flowchart showing an example of a flow of processing of the road surface damage detection device 10A and the processing is executed on the vehicle 18 side. FIG. 13 is a flowchart showing an example of a flow of processing executed on the road information providing system 14A side.

First, as shown in the flowchart of FIG. 12, the information processing unit 52 reads the vehicle information at a predetermined processing cycle during travelling of the vehicle (S500). The vehicle information includes, for example, the position information indicating the current position of the vehicle 18 and acquired by the position information acquisition unit 20, the sensor information acquired by the front-rear acceleration acquisition unit 22, that is, the front-rear directional acceleration indicating the road surface damage, the image information (the captured image) acquired by the image acquisition unit 28 and corresponding to the position indicated by the position information and to the position of the road surface damage indicated by the sensor information. As described above, the image information corresponding to the position of the road surface damage is mainly for facilitating visual understanding of the degree and position of the road surface damage, and reading of the image information may be omitted. Further, the information processing unit 52 associates the position information acquired by the position information acquisition unit 20, the sensor information acquired by the front-rear acceleration acquisition unit 22, and the image information (the captured image) acquired by the image acquisition unit 28 one another based on the time stamps attached to the respective pieces of information to generate the road surface damage related information (S502). Further, the information processing unit 52 transmits the generated road surface damage related information to the road information providing system 14A side via the transmission unit 26a (S504).

As shown in the flowchart of FIG. 13, the information collection unit 42 of the road information providing system 14A monitors whether the reception unit 40a receives the road surface damage related information (the position information, the sensor information, and the captured image) from the vehicle 18 (No in S600). Further, when the road surface damage related information is received (Yes in S600), the information collection unit 42 collects the road surface damage related information, and sequentially supplies the received information to the damage state acquisition unit 54. As described with reference to FIG. 5, the occurrence rate calculation unit 54b of the damage state acquisition unit 54 calculates the number of acquisition times n of the sensor information while the vehicle advances for the predetermined travel distance L (S602). The road surface damage detection unit 54a, while the vehicle advances for the predetermined travel distance L, calculates the number of times of detection of the road surface damage (S604), and calculates the occurrence rate p₍ₜ₎ of the road surface damage (S606).

The damage state acquisition unit 54 generates the road surface damage information P_{c(t)}=[p₍ₜ₎, x₍ₜ₎, y₍ₜ₎] by associating the occurrence rate p₍ₜ₎ of the road surface damage at the current time point t with the position coordinates (x₍ₜ₎, y₍ₜ₎) on the map (S608). When generating the road surface damage information P_{c(t)}, the damage state acquisition unit 54 associates the captured image corresponding to the position of the road surface damage with the road surface damage information P_{c(t)}. When the road surface damage information P_{c(t)} is generated, the information construction unit 44 updates the road surface state database 50 (S610).

Similarly as the road information providing system 14, the information construction unit 44 executes processing in accordance with the flowchart shown in FIG. 9. That is, when a plurality of pieces of road surface damage related information in a predetermined region on the map is provided from a single vehicle 18 or a plurality of pieces of road surface damage related information in a predetermined region on the map is provided from a plurality of vehicles 18, the information construction unit 44 further executes data construction for improving the accuracy of the road surface damage information in the region. For example, as shown in FIG. 9, the information construction unit 44 extracts all road surface damage information P_{c(t)} belonging to the coordinates (X, Y) on the map from the plurality of pieces of road surface damage information P_{c(t)} generated by the damage state acquisition unit 54 (S300). The information construction unit 44 calculates the occurrence rate P(X, Y) of the road surface damage at the coordinates (X, Y) on the map in accordance with Formula 3 described above (S302). Further, the information construction unit 44 constructs the road surface damage information at the coordinates (X, Y) in the road surface state database 50 based on the plurality of pieces of road surface damage information P_{c(t)} (S304). As a result, the accuracy of the road surface damage information at the coordinates (X, Y) can be improved. When constructing the road surface state database 50, the information construction unit 44 may rank the degree of the road surface damage and attach rank information. For example, when the road surface damage information is provided to the driver of the vehicle 18 or the road manager RM by displaying information on the map, for example, color coding may be performed for each rank so as to be displayed. An alert mark or the like in accordance with the rank may be displayed. That is, the “danger level” corresponding to the road surface damage may be displayed.

As described above, the road surface damage detection device 10 according to the embodiment includes, for example, the front-rear acceleration acquisition unit 22, the position information acquisition unit 20, the damage state acquisition unit 24, and the transmission unit 26a. The front-rear acceleration acquisition unit 22 acquires the front-rear directional acceleration of the vehicle 18 using, for example, an existing acceleration sensor during travelling of the vehicle 18. The position information acquisition unit 20 acquires the position information indicating the current position of the vehicle 18 based on, for example, a signal from the GPS or information acquired by a navigation system. The damage state acquisition unit 24 acquires the road surface damage information which is related to the road surface on which the vehicle 18 travels and in which the road surface damage of the road surface detected based on a change state of the front-rear directional acceleration and the position information corresponding to a position where the road surface damage is detected is associated with each other. Further, the transmission unit 26a transmits the acquired road surface damage information to an outside the vehicle, for example, to the road information providing system 14.

According to the road surface damage detection device 10 described above, for example, when the wheel 38 of the vehicle 18 passes through a damaged portion of the road surface during travelling, the wheel 38 is caught for a moment and jumps over the damaged portion. At this time, an acceleration in a front-rear direction of the vehicle 18 is generated. A change in the front-rear directional acceleration is less likely to be affected by a measurement environment (for example, weather or ambient brightness), and the change depends on a magnitude of the road surface damage. Therefore, by associating the change state of the front-rear directional acceleration with the position information at that time, it is possible to acquire highly accurate road surface damage information that can specify the presence or absence of the road surface damage and the position where the road surface damage occurs. Further, the acquired highly accurate road surface damage information can be provided to the user outside the vehicle, for example, drivers of other vehicles, and the road manager RM.

The road surface damage detection device 10 may further include, for example, the reception unit 26b that receives the road surface damage information related to the road surface in the travelling direction of the vehicle 18. According to the configuration, for example, the road surface damage information which is already acquired by other vehicles and which is related to a road surface on which the vehicle 18 (own vehicle) is going to travel can be received, and the vehicle control and driver alert can be performed in accordance with the road surface state. The road in the travelling direction of the vehicle 18 (own vehicle) which is a reception target of the road surface damage information can be specified by, for example, input of the driver or a guide road to a destination presented by a navigation device.

The road surface damage detection device 10 may further include, for example, the image acquisition unit 28 that acquires a captured image from the image capturing unit 28a that captures an image of the road surface during travelling of the vehicle 18. Further, the transmission unit 26a may associate the road surface damage information and the captured image corresponding to the position where the road surface damage is detected, and transmit the information. According to the configuration, for example, a degree of the road surface damage can be provided to the external user, for example, drivers of other vehicles or the road manager RM, in a form that the degree of the road surface damage information is easy to be understood visually, and the position of the road surface damage can be more easily understood from a surrounding condition reflected along with a road surface condition.

The damage state acquisition unit 24 of the road surface damage detection device 10 may include, for example, the occurrence rate calculation unit 24b that calculates the occurrence rate of the road surface damage for a predetermined travelled range. In this case, the transmission unit 26a may transmit the calculated occurrence rate together with the road surface damage information. According to the configuration, for example, when the road surface damage information is provided to drivers of other vehicles, the road surface damage information can be provided as a road range to be alerted during travelling, and information that is easy to handle can be provided. When the road surface damage information is provided to the road manager RM, a road range requiring repair (mend) can be presented, and the necessity of repair and the range can be easily determined.

The information sharing system 16 according to the embodiment includes the information collection unit 42, the information construction unit 44, and the information providing unit 46. The information collection unit 42 collects, for example, the road surface damage information transmitted from the road surface damage detection device 10, and the information construction unit 44 constructs the road surface state database 50 indicating the road surface state based on the collected road surface damage information. The information providing unit 46 selects, from the road surface state database 50, the road surface damage information related to a road requested by the user such as the driver or the road manager RM, and provides the road surface damage information to the user. According to the configuration, for example, the road surface state database 50 can be constructed based on a plurality of pieces of road surface damage information acquired when the vehicle 18 actually travels on the road. As a result, the road surface damage information with higher accuracy can be provided to the external user, for example, drivers of other vehicles and the road manager RM. In this case, since the generation of the road surface damage information is executed on the vehicle 18 side, a processing load on the road information providing system 14 side can be reduced.

The road information providing system 14A according to the embodiment includes, for example, the information collection unit 42, the damage state acquisition unit 54, the information construction unit 44, and the information providing unit 46. The information collection unit 42 collects information which is transmitted from the vehicle 18 and in which the front-rear directional acceleration acquired during travelling of the vehicle 18 and the position information indicating the position of the vehicle 18 when the acceleration is acquired is associated with each other. The information may be associated with each other on the road surface damage detection device 10 side or on the road information providing system 14A side. The damage state acquisition unit 54 acquires the road surface damage information in which the road surface damage of the road surface detected based on a change state of the front-rear directional acceleration and the position information corresponding to a position where the road surface damage is detected is associated with each other. The information construction unit 44 constructs the road surface state database 50 indicating the road surface state based on the acquired road surface damage information. The information providing unit 46 selects, from the road surface state database 50, the road surface damage information related to a road requested by the user such as the driver of the vehicle 18 or the road manager RM, and provide the selected road surface damage information to the user.

According to the road information providing system 14A described above, for example, since the road surface state database 50 can be constructed based on a plurality of pieces of road surface damage information acquired when the vehicle 18 actually travels on the road, the road surface damage information with higher accuracy can be provided to the external user such as drivers of other vehicles or the road manager RM. In this case, since the generation of the road surface damage information is executed on the road information providing system 14A side, the processing load on the vehicle 18 side can be reduced, and therefore the road surface damage detection device on the vehicle 18 side can be easily introduced.

The information collection unit 42 of the road information providing system 14A may further acquire, for example, a captured image from the image capturing unit 28a that captures an image of the road surface during travelling of the vehicle 18. Further, the information providing unit 46 may associate the road surface damage information and the captured image corresponding to the position where the road surface damage is detected, and provide the information to the user. According to the configuration, for example, the degree of the road surface damage can be provided to the external user, for example, the driver of the vehicle 18 or the road manager RM, in a form that the degree of the road surface damage is easy to be understood visually, and the position of the road surface damage can be more easily understood from a surrounding condition reflected along with a road surface condition.

The damage state acquisition unit 54 of the road information providing system 14A may include, for example, the occurrence rate calculation unit 54b that calculates the occurrence rate of the road surface damage in a predetermined range on the map data. Further, the information providing unit 46 may provide the occurrence rate together with the road surface damage information to the external user, for example, the driver of the vehicle 18 or the road manager RM. According to the configuration, for example, when the road surface damage information is provided to the driver of the vehicle 18, the road surface damage information can be provided as a road range to be alerted during travelling, and information that is easy to handle can be provided. When the road surface damage information is provided to the road manager RM, a road range requiring repair (mend) can be presented, and the necessity of repair and the range can be easily determined.

The program for implementing each functional module of the road surface damage detection device 10 (the road surface damage detection device 10A) and the road information providing system 14 (the road information providing system 14A) as described above may be provided in a form of being recorded in a computer-readable recording medium such as a CD-ROM, a flexible disk (FD), a CD-R, and a digital versatile disk (DVD) in a format that the program can be installed and executed.

Further, the program may be stored on a computer connected to a network such as the Internet, and provided by being downloaded via the network. The program executed in the present embodiment may be provided or distributed via a network such as the Internet.

A road surface damage detection device according to an aspect of this disclosure includes, for example, a front-rear acceleration acquisition unit configured to acquire a front-rear directional acceleration of a vehicle during travelling of the vehicle; a position information acquisition unit configured to acquire position information indicating a current position of the vehicle; a damage state acquisition unit configured to acquire road surface damage information, which is related to a road surface on which the vehicle travels and in which road surface damage of the road surface detected based on a change state of the acceleration, and the position information corresponding to a position where the road surface damage is detected are associated with each other; and a transmission unit configured to transmit the acquired road surface damage information to an outside of the vehicle. According to the configuration, for example, when a wheel of the vehicle passes through a damaged portion of the road surface during travelling, the wheel is caught for a moment and jumps over the damaged portion. At this time, an acceleration in a front-rear direction of the vehicle is generated. A change in the front-rear directional acceleration is less likely to be affected by a measurement environment (for example, weather or ambient brightness), and the change depends on a magnitude of the road surface damage. Therefore, by associating the change state of the front-rear directional acceleration with the position information at that time, it is possible to acquire highly accurate road surface damage information that can specify presence or absence of the road surface damage and the position where the road surface damage occurs. Further, the acquired highly accurate road surface damage information can be provided to a user outside the vehicle, for example, drivers of other vehicles, and a road manager.

The road surface damage detection device according to the aspect of this disclosure may further include, for example, a reception unit configured to receive the road surface damage information related to the road surface in a travelling direction of the vehicle. According to the configuration, for example, the road surface damage information which is already acquired by other vehicles and which is related to a road surface on which an own vehicle is going to travel can be received, and vehicle control and driver alert can be performed in accordance with the road surface state. The road in the travelling direction of the own vehicle which is a reception target of the road surface damage information can be specified by, for example, input of the driver or a guide road to a destination presented by a navigation device.

The road surface damage detection device according to the aspect of this disclosure may further includes, for example, an image acquisition unit configured to acquire a captured image from an image capturing unit configured to capture an image of the road surface during travelling of the vehicle, and the transmission unit may transmit the road surface damage information and the captured image corresponding to the position where the road surface damage is detected in association with each other. According to the configuration, for example, a degree of the road surface damage can be provided to an external user in a form that the degree of the road surface damage is easy to be understood visually, and the position of the road surface damage can be more easily understood from a surrounding condition reflected along with a road surface condition.

The damage state acquisition unit of the road surface damage detection device according to the aspect of this disclosure may include, for example, a road surface damage detection unit configured to detect presence or absence of the road surface damage by analyzing the front-rear directional acceleration of the vehicle acquired by the front-rear acceleration acquisition unit; and an occurrence rate calculation unit configured to calculate an occurrence rate of the road surface damage for a predetermined travelled range, and the transmission unit may transmit the occurrence rate together with the road surface damage information. According to the configuration, for example, when the road surface damage information is provided to drivers of other vehicles, the road surface damage information including the occurrence rate of the road surface damage can be provided as a road range to be alerted during travelling, and information that is easy to handle can be provided. When the road surface damage information is provided to the road manager, a road range (for example, a range having a high occurrence rate of the road surface damage) requiring repair (mend) can be presented, and the necessity of repair and the range can be easily determined.

A road information providing system according to another aspect of this disclosure includes, for example, an information collection unit configured to collect the road surface damage information transmitted from the road surface damage detection device; an information construction unit configured to construct a road surface state database indicating a road surface state based on the collected road surface damage information; and an information providing unit configured to select, from the road surface state database, the road surface damage information related to a road requested by a user, and provide the selected road surface damage information to the user. According to the configuration, for example, since the road surface state database can be constructed based on a plurality of pieces of road surface damage information acquired when the vehicle actually travels on the road, the road surface damage information with higher accuracy can be provided to the external user such as drivers of other vehicles or a road manager. In this case, since generation of the road surface damage information is executed on the vehicle side, a processing load on the road information providing system side can be reduced.

A road information providing system according to another aspect of this disclosure includes, for example, an information collection unit configured to collect information which is transmitted from a vehicle and in which a front-rear directional acceleration acquired during travelling of the vehicle on a road surface and position information indicating a position of the vehicle when the acceleration is acquired is associated with each other; a damage state acquisition unit configured to acquire road surface damage information in which road surface damage of the road surface detected based on a change state of the acceleration and the position information corresponding to a position where the road surface damage is detected is associated with each other; an information construction unit configured to construct a road surface state database indicating a road surface state based on the acquired road surface damage information; and an information providing unit configured to select, from the road surface state database, the road surface damage information related to a road requested by a user, and provide the selected road surface damage information to the user. According to the configuration, for example, since the road surface state database can be constructed based on a plurality of pieces of road surface damage information acquired when the vehicle actually travels on the road, the road surface damage information with higher accuracy can be provided to the external user such as drivers of other vehicles or a road manager. In this case, since the generation of the road surface damage information is executed on the road information providing system side, a processing load on the vehicle side can be reduced, and therefore the road surface damage detection device on the vehicle side can be easily introduced.

The information collection unit of the road information providing system according to the aspect of this disclosure may further acquire, for example, a captured image from an image capturing unit configured to capture an image of the road surface during travelling of the vehicle, and the information providing unit may provide the road surface damage information and the captured image corresponding to the position where the road surface damage is detected in association with each other. According to the configuration, for example, a degree of the road surface damage can be provided to the external user in a form that the degree of the road surface damage is easy to be understood visually, and the position of the road surface damage can be more easily understood from a surrounding condition reflected along with a road surface condition.

The damage state acquisition unit of the road information providing system according to the aspect of this disclosure may include, for example, a road surface damage detection unit configured to detect presence or absence of the road surface damage by analyzing a front-rear directional acceleration of the vehicle acquired by the information collection unit; and an occurrence rate calculation unit configured to calculate an occurrence rate of the road surface damage for a predetermined range on map data, and the information providing unit may provide the occurrence rate together with the road surface damage information. According to the configuration, for example, when the road surface damage information is provided to drivers of other vehicles, the road surface damage information including the occurrence rate of the road surface damage can be provided as a road range to be alerted during travelling, and information that is easy to handle can be provided. When the road surface damage information is provided to the road manager, a road range (for example, a range having a high occurrence rate of the road surface damage) requiring repair (mend) can be presented, and the necessity of repair and the range can be easily determined.

Although the embodiment and the modification disclosed here are described, the embodiment and the modification are presented as examples and are not intended to limit the scope of this disclosure. These new embodiments can be implemented in other various forms, and various omissions, replacements, and changes can be made without departing from the spirit of this disclosure. These embodiments and modifications thereof are included in the scope and gist of this disclosure, and are also included in the inventions described in the claims and equivalents thereof.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A road surface damage detection device comprising:
a front-rear acceleration acquisition unit configured to acquire a front-rear directional acceleration of a vehicle during travelling of the vehicle;
a position information acquisition unit configured to acquire position information indicating a current position of the vehicle;
a damage state acquisition unit configured to acquire road surface damage information which is related to a road surface on which the vehicle travels and in which road surface damage of the road surface detected based on a change state of the acceleration, and the position information corresponding to a position where the road surface damage is detected are associated with each other; and
a transmission unit configured to transmit the acquired road surface damage information to an outside of the vehicle.

2. The road surface damage detection device according to claim 1, further comprising:
a reception unit configured to receive the road surface damage information related to the road surface in a travelling direction of the vehicle.

3. The road surface damage detection device according to claim 1, further comprising:
an image acquisition unit configured to acquire a captured image from an image capturing unit configured to capture an image of the road surface during travelling of the vehicle, wherein
the transmission unit transmits the road surface damage information and the captured image corresponding to the position where the road surface damage is detected in association with each other.

4. The road surface damage detection device according to claim 1, wherein
the damage state acquisition unit includes:
a road surface damage detection unit configured to detect presence or absence of the road surface damage by analyzing the front-rear directional acceleration of the vehicle acquired by the front-rear acceleration acquisition unit; and
an occurrence rate calculation unit configured to calculate an occurrence rate of the road surface damage for a predetermined travelled range, wherein
the transmission unit transmits the occurrence rate together with the road surface damage information.

5. A road information providing system comprising:
an information collection unit configured to collect the road surface damage information transmitted from at least one road surface damage detection device according to claim 1;
an information construction unit configured to construct a road surface state database indicating a road surface state based on the collected road surface damage information; and
an information providing unit configured to select, from the road surface state database, the road surface damage information related to a road requested by a user, and provide the selected road surface damage information to the user.

6. A road information providing system comprising:
an information collection unit configured to collect information which is transmitted from a vehicle and in which a front-rear directional acceleration acquired during travelling of the vehicle on a road surface and position information indicating a position of the vehicle when the acceleration is acquired is associated with each other;
a damage state acquisition unit configured to acquire road surface damage information in which road surface damage of the road surface detected based on a change state of the acceleration and the position information corresponding to a position where the road surface damage is detected is associated with each other;
an information construction unit configured to construct a road surface state database indicating a road surface state based on the acquired road surface damage information; and
an information providing unit configured to select, from the road surface state database, the road surface damage information related to a road requested by a user, and provide the selected road surface damage information to the user.

7. The road information providing system according to claim 6, wherein
the information collection unit further acquires a captured image from an image capturing unit configured to capture an image of the road surface during travelling of the vehicle, and
the information providing unit provides the road surface damage information and the captured image corresponding to the position where the road surface damage is detected in association with each other.

8. The road information providing system according to claim 6, wherein
the damage state acquisition unit includes:
a road surface damage detection unit configured to detect presence or absence of the road surface damage by analyzing a front-rear directional acceleration of the vehicle acquired by the information collection unit; and
an occurrence rate calculation unit configured to calculate an occurrence rate of the road surface damage for a predetermined range on map data, wherein
the information providing unit provides the occurrence rate together with the road surface damage information.
